(19) United States
(12) Patent Application Publication
(10) Pub. No.: US 2020/0048608 A1
(43) Pub. Date: Feb. 13, 2020

(54) METHOD FOR IN VITRO ACTIVATION OF IMMUNE CELLS

(71) Applicant: Lifenergy Biotech Corp., Taipei (TW)

(72) Inventor: YI-SHYANG HUANG, Taipei (TW)

(21) Appl. No.: 16/538,854

(22) Filed: Aug. 13, 2019

Related U.S. Application Data
(60) Provisional application No. 62/718,371, filed on Aug. 13, 2018.
(60) Provisional application No. 62/741,539, filed on Oct. 5, 2018.

(51) Int. Cl. C12N 5/0783; A61K 35/17

(52) U.S. Cl. CPC C12N 5/0646; A61K 35/17; C12N 2501/2302; C12N 2501/90; C12N 2501/2315; C12N 2501/2318; C12N 2501/2321; C12N 2501/2312

(57) ABSTRACT

The present disclosure provides a method for in vitro activation of immune cells for immune cell therapies. The method includes contacting a population of immune cells with β-glucan to obtain a population of conditioned cells. When introduced into a subject, the population of conditioned immune cells inhibits tumor growth in the subject. A method for inhibiting tumor growth using the population of conditioned immune cells is also provided.

### CROSS-REFERENCE TO RELATED APPLICATION

The present disclosure claims the benefit of U.S. provisional application No. 62/718371, filed on Aug. 13, 2018, and U.S. provisional application No. 62/741539, filed on Oct. 5, 2018, the entirety of which is incorporated herein by reference

### FIELD OF THE DISCLOSURE

The present disclosure relates to a method for in vitro activation of immune cells, and more particularly, to a method for inhibiting tumor growth using in vitro-activated immune cells.

### BACKGROUND OF THE DISCLOSURE

Immune cell therapy, also known as adoptive cell transfer, is a cancer treatment that involves infusion of various immune cell subsets to eliminate tumors and preventing cancer recurrence. Typically, an immune cell therapy is performed by isolating immune cells from an individual, expanding, activating and/or genetically modifying the immune cells in vitro, and returning the expanded, activated or modified immune cells to the same or another individual. For example, at autologous T cell therapies, chimeric antigen receptors (CARs) or cancer target-specific T cell receptors (TCRs) are transduced and expressed in a patient's T cells in vitro to render the T cells tumor specificity before reinfusing the genetically engineered T cells back into the patient.

However, effectiveness of existing immune cell therapies may vary and is often limited. Therefore, there is a need for a method that enhances treatment effectiveness of immune cell therapies.

### BRIEF SUMMARY OF THE DISCLOSURE

An objective of the present disclosure is to provide a method for in vitro activation and pre-infusion expansion of immune cells.

Another objective of the present disclosure is to provide a population of activated immune cells for enhancing effectiveness of immune cell therapies.

An embodiment of the present disclosure provides a method for in vitro activation of immune cells. The method includes contacting a population of immune cells with β-glucan to obtain a population of conditioned immune cells. When introduced into a subject, the population of conditioned immune cells inhibits tumor growth in the subject.

Preferably, adaptive immunity induced by the population of conditioned immune cells is stronger than that induced by the non-conditioned population of immune cells.

Preferably, the population of immune cells comprise natural killer (NK) cells.

Preferably, the β-glucan comprises glucose monomers organized as β-(1,3)-linked glucopyranose backbone with periodic β-(1,3) glucopyranose branches linked to the backbone via β-(1,6) glycosidic linkages.

Preferably, the β-glucan is extracted from Saccharomyces cerevisiae

Preferably, a concentration of the β-glucan falls within a range of 40 μg/ml to 4 mg/ml.

Preferably, the population of immune cells are contacted with the β-glucan for a duration of 12-15 days.

Preferably, the method further includes contacting the population of immune cells with at least one cylokine.

Preferably, the cytokine includes interleukin-15 (IL-15), interleukin-2 (IL-2), interleukin-12 (IL-12), interleukin-18 (IL-18), and interleukin-21 (IL-21).

Another embodiment of the present disclosure provides a method of inhibiting tumor growth in a subject. The method includes administering a therapeutically effective amount of the aforementioned population of conditioned immune cells to the subject.

Yet another embodiment of the present disclosure provides a population of β-glucan conditioned immune cells for inhibition of tumor growth in a subject.

### BRIEF DESCRIPTION OF THE DRAWINGS

The accompanying drawings illustrate one or more embodiments of the present invention and, together with the written description, explain the principles of the present invention. Wherever possible, the same reference numbers are used throughout the drawings referring to the same or like elements of an embodiment.

FIG. 1 is a schematic diagram of a timeline for pre-infusion expansion of NK cells in accordance with an embodiment of the present disclosure;

FIG. 2 is a schematic diagram of a timeline for infusion of BM-NK cells and conditioned BM-NK cells prepared according to the scheme of FIG. 1 in accordance with an embodiment of the present disclosure;

FIG. 3 is a curve diagram showing the time-dependent changes in tumor size in mice treated according to the scheme of FIG. 2 in accordance with an embodiment of the present disclosure; and

FIGS. 4, 5 and 6 are results of flow cytometry analyses of splenocytes of the mice treated according to the scheme of FIG. 2 in accordance with an embodiment of the present disclosure.

### DETAILED DESCRIPTION OF THE PREFERRED EMBODIMENTS

The present invention will now be described more fully hereinafter with reference to the accompanying drawings illustrating various exemplary embodiments of the invention. The present invention may, however, be embodied in many different forms and should not be construed as limited to the embodiments set forth herein. Rather, these embodiments are provided so that this disclosure will be thorough and complete, and will fully convey the scope of the disclosure to those skilled in the art. Like reference numerals refer to like elements throughout.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

It will be understood that the terms “and/or” and “at least one” include any and all combinations of one or more of the associated listed items. It will also be understood that, although the terms first, second, third etc. may be used herein to describe various elements, components, regions, parts and/or sections, these elements, components, regions, parts and/or sections should not be limited by these terms. These terms are only used to distinguish one element, component, region, part or section from another element, component, region, layer or section. Thus, a first element, component, region, part or section discussed below could be termed a second element, component, region, layer or section without departing from the teachings of the present disclosure.

The term “β-glucan” used herein refers to soluble or particulate polysaccharides extracted from Saccharomyces cerevisiae and composed of glucose monomers organized as β-(1,3)-linked glucopyranose backbone with periodic β-(1,3) glucopyranose branches linked to the backbone via β-(1,6) glycosidic linkages. The soluble β-glucan described in various embodiments of the present disclosure has a molecular weight of roughly 120-205 kDa. The particulate β-glucan described in various embodiments of the present disclosure has a diameter of roughly 2-4 μm.

The term “immune cell therapy” used herein refers to cancer vaccines or therapies that involves transfusion of cytokine-induced killer (CIK) cells, natural killer (NK) cells, dendritic cells (DC), DC-CIK cells, gammadelta T cells, genetically engineered CAR-T cells, genetically engineered TCR T cells, autologous tumor infiltrating lymphocytes (TIL), and/or genetically re-directed peripheral blood mononuclear cells.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

In an aspect of the present disclosure, β-glucan is used in combination with one or more immune cell therapies to treat a variety of cancers. Such variety of cancers include, but are not limited to, unresectable or metastatic (advanced) melanoma, colorectal cancer, gastric cancer, metastatic non-small cell lung cancer (NSCLC), recurrent or metastatic squamous cell carcinoma of the head and neck (SCCHN), classical Hodgkin lymphoma (cHL), locally advanced or metastatic urothelial carcinoma, solid tumor cancers expressing biomarker microsatellite instability-high (MSI-H) or mismatch repair deficient (dMMR), metastatic renal cell carcinoma, hepatocellular carcinoma (HCC), metastatic Merkel cell carcinoma (MCC), and other types of carcinoma of the skin, lung, kidney, bladder, head and neck, liver, breast and other organs of the body, as well as leukemia, multiple myeloma, and other types of cancers of the circulatory systems.

In at least one embodiment, β-glucan may modulate the immunosuppressed tumor microenvironment and/or promote mobilization or infiltration of activated immune cells to the site of tumor. In other embodiments, β-glucan may promote in vitro expansion and/or efficiency of immune cells. In other words, β-glucan produces a synergistic effect with the combined immune cell therapy in treatment of cancer.

Another aspect of the present disclosure pertains to methods for treating a subject suffering from or susceptible to one or more of a variety of cancers. Such cancers may include unresectable or metastatic (advanced) melanoma, colorectal cancer, gastric cancer, metastatic non-small cell lung cancer (NSCLC), recurrent or metastatic squamous cell carcinoma of the head and neck (SCCHN), classical Hodgkin lymphoma (cHL), locally advanced or metastatic urothelial carcinoma, solid tumor cancers expressing biomarker microsatellite instability-high (MSI-H) or mismatch repair deficient (dMMR), metastatic renal cell carcinoma, hepatocellular carcinoma (HCC), metastatic Merkel cell carcinoma (MCC), and other types of carcinoma of the skin, lung, kidney, bladder, head and neck, liver, breast and other organs of the body, as well as leukemia, multiple myeloma, and other types of cancers of the circulatory systems.

In an embodiment, the method includes administering to the subject β-glucan in combination with one or more immune cell therapies. The administered β-glucan is preferably in a therapeutically or prophylactically effective amount sufficient to modulate the immunosuppressed tumor microenvironment and/or promote mobilization or infiltration of activated immune cells. In other words, β-glucan is administered to the subject in an amount sufficient to produce a synergistic effect with the combined immune cell therapy.

A “therapeutically effective amount” refers to an amount effective, at dosages and for periods of time necessary, to achieve the desired therapeutic result, such as suppression or inhibition of tumor growth. A therapeutically effective amount of β-glucan may vary according to factors such as the disease stage, age, gender, and weight of the subject, and the ability of β-glucan to elicit a desired response in the subject. Dosage regimens may be adjusted to provide the optimum therapeutic response. A therapeutically effective amount is also one in which any toxic or detrimental effects of β-glucan are outweighed by the therapeutically beneficial effects.

A “prophylactically effective amount” refers to an amount effective, at dosages and for periods of time necessary, to achieve the desired prophylactic result, such as preventing or inhibiting metastasis of a tumor. A prophylactically effective amount can be determined as described above for the therapeutically effective amount. Typically, since a prophylactic dose is used in subjects prior to or at an earlier stage of disease, the prophylactically effective amount shall be higher than the therapeutically effective amount.

It is to be noted that dosages of β-glucan may vary with the severity of the condition to be alleviated. It is to be further understood that for any particular subject, specific dosage regimens should be adjusted over time according to the individual need and the professional judgment of the person administering or supervising the administration of the immunotherapeutic combination.

β-Glucan may be administered in a time release formulation, for example in a composition which includes a slow release polymer, or may be prepared with carriers that would protect β-glucan against rapid release, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Yet another aspect of the present disclosure pertains to methods for in vitro activation of immune cells for immune cell therapies. In an embodiment, the method includes contacting a population of immune cells with β-glucan to obtain a population of conditioned immune cells. The immune cells may include, but are not limited to, NK cells, CIK cells and DC-NIK cells. The NK cells may be obtained from peripheral blood. In some embodiments, the NK cells may be derived from precursors, such as hematopoietic stem cells (HSCs) and lymphoid progenitors, obtained from bone marrow or peripheral blood. The NK cells may be an immortalized NK cell line, for example, NK-92. In other embodiments, the NK cells may be derived from induced pluripotent stem cells (iPSC).

In the embodiment, the amount of β-glucan contacted with the population of immune cells is sufficient to promote in vitro activation and expansion of the immune cells. Specifically, a concentration of the β-glucan contacting with the population cells may fall within a range of 40 μg/ml-4 mg/ml, and the population of immune cells may be contacted with β-glucan for a duration of 12-15 days prior to infusion into a subject in need. In the embodiment, the population of immune cells may be autologous or allogeneic.

In an embodiment, the method for in vitro activation of immune cells may further include a step of genetically modifying the population of conditioned immune cells. Specifically, the genetic modification may include, but is not limited to, transduction with chimeric antigen receptor genes.

In an embodiment, the method for in vitro activation of immune cells may further include a step of contacting the population of immune cells with at least one cytokine. The cytokine may include, but is not limited to, interleukin-15 (IL-15), interleukin-2 (IL-2), interleukin-12 (IL-12), interleukin-18 (IL-18), and interleukin-21 (IL-21). Specifically, a concentration of the cytokine contacting the population of immune cell may be sufficient to promote in vitro expansion and/or activation of the immune cells; and the population of immune cells may be contacted with the cytokine for a duration sufficient to allow the immune cell proliferate to a desire population. In an example, 10-50 ng/ml of IL-15 may be used in combination with 40 μg/ml-4 mg/ml of β-glucan for treating NK cells for 12-15 days. In the embodiment, the population of immune cells may be contacted with the cytokine before or after contacted with β-glucan; alternatively, β-glucan and cytokine may be used simultaneously to treat the population of immune cells prior to infusion.

Another aspect of the present disclosure provides a method of inhibiting tumor growth in a subject. In an embodiment, the method includes administering to the subject a therapeutically effective amount of the population of conditioned immune cells obtained according to the aforementioned in vitro immune cell activation method. The therapeutically effective amount administered to the subject may range from 2×10⁷ to 5×10⁸ cells/injection. The conditioned immune cells may be administered through dorsal subcutaneous injection.

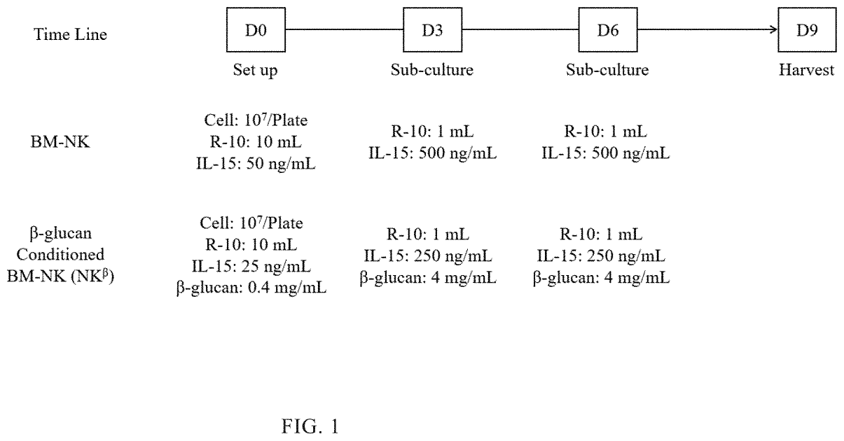

Referring now to FIG. 1, a timeline for pre-infusion expansion of NK cells in accordance with an embodiment of the present disclosure is provided. As shown FIG. 1, on day 0 (D0) of the experiment, 1×10⁷/plate of bone marrow cells were seeded in 10 mL of RPM1 1640 with 10% serum (R-10) medium: the bone marrow cells were divided into two groups, one being treated with 50 ng/mL of IL-15 (denoted as BM-NK group) while the other being treated with 25 ng/mL of IL-15 and 0.4 mg/mL of β-glucan (denoted as conditioned BM-NK group). The cells were incubated at 37° C. under 5% CO₂. On day 3 (D3) and day 6 (D6), the BM-NK group was subcultured in 1 mL of R-10 supplemented with 500 ng/mL of IL-15, whereas the conditioned BM-NK group was subcultured in 1 mL of R-10 supplemented with 250 ng/mL of IL-15 and 4 mg/mL of β-glucan. The cells were harvested on day 9 (D9) for infusion.

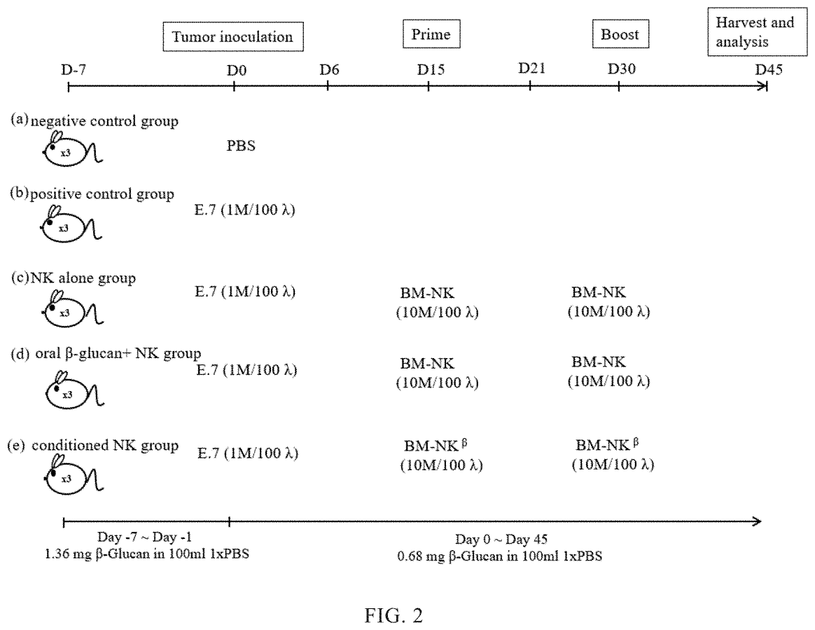

Referring now to FIG. 2, a timeline for infusion of the BM-NK cells and conditioned BM-NK cells prepared according to the scheme shown in FIG. 1 is provided. A total of fifteen mice were divided into a negative control group, a positive control group, a NK alone group, an oral β-glucan+NK group and a conditioned NK group. Prior to tumor inoculation, each of the three mice in the oral β-glucan+NK group was gavaged with 1.36 mg/mL of β-glucan dissolved in 100 ml 1×PBS daily for 7 consecutive days. On day 0 of tumor inoculation, 1×10⁶ E.G7-OVA cells (E.7) were subcutaneously injected into one lateral flank of each of the mice in all groups, except for the negative control group. Fifteen days (D15) and thirty days (D30) after tumor inoculation, 1×10⁷ BM-NK cells were subcutaneously injected into a contralateral flank of each of the mice in the NK alone and oral β-glucan+NK groups; similarly, 1×10⁷ β-glucan conditioned BM-NK cells were subcutaneously injected into a contralateral flank of each of the mice in the conditioned NK group. Meanwhile, each of the three mice in the oral β-glucan+NK group was gavaged with 6.8 mg/mL of β-glucan dissolved in 100 ml 1×PBS daily after tumor inoculation. Tumor volumes of the mice are measured every 2 to 5 days. All of the mice were sacrificed and analyzed on day 45.

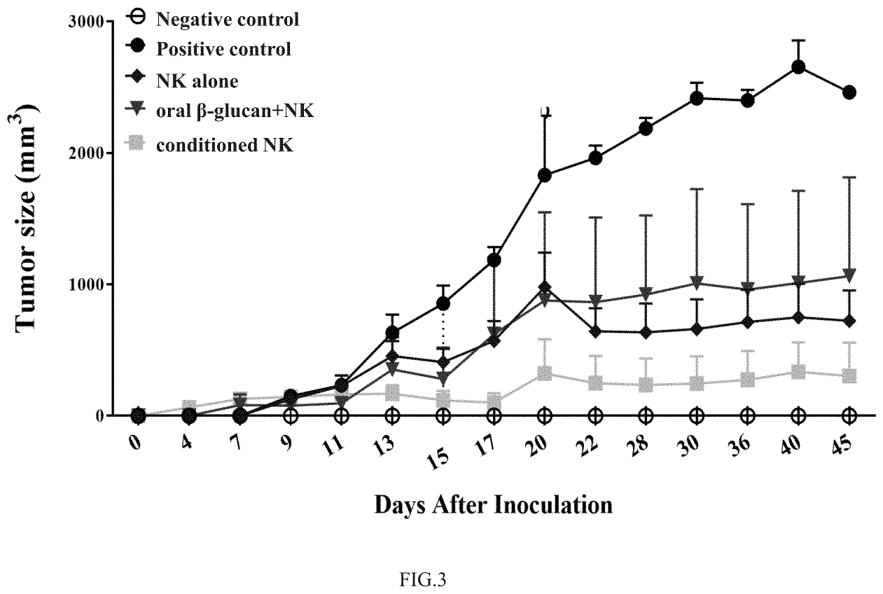

Referring now to FIG. 3, a curve diagram showing the time-dependent changes in tumor size in the groups of mice treated according to the scheme shown in FIG. 2 is provided. As shown in FIG. 3, when compared to the positive control group, all of the experimental groups exhibited significant tumor reduction and/or inhibition of tumor growth. Specifically, the conditioned NK group exhibited significantly lowered tumor size as compared with the NK alone and oral β-glucan+NK groups. The results suggest that the pre-infusion β-glucan treatment exemplified in FIG. 1 can effectively activate NK cells and therefore inhibit tumor growth.

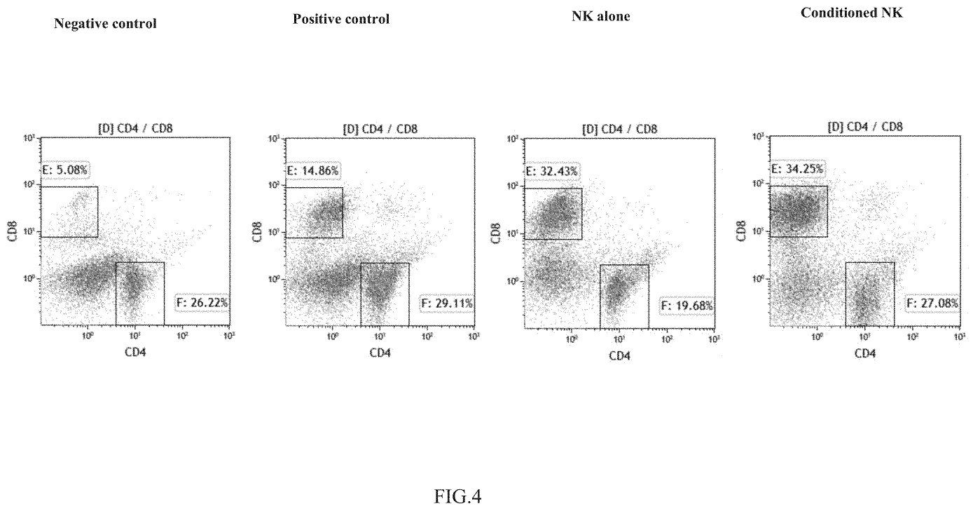

Referring now to FIG. 4, results of a flow cytometry analysis of splenocytes of the mice are provided. Specifically, spleens of the mice in the indicated groups are removed and prepared for single cell suspensions after the mice were sacrificed. The obtained splenocytes were then labeled with carboxyfluorescein succinimidyl ester (CFSE) and re-stimulated with cognate MHC 1-restricted OVA₂₅₇₋₂₆₄ for 5 days. Percentages of lineage-TCR_{ab}+CD4⁺ T cells and CD8⁺ T cells were determined and shown on dot plots by using specific antibodies and flow cytometry. As shown in FIG. 4, the percentage of CD8⁺ T cells from the NK alone group was 32.43%, whereas the percentage of CD8⁺ T cells from the conditioned NK group was increased to 34.25%. In other words, pre-infusion in-vitro treatment of NK cells with β-glucan may cause enhancement of the cytotoxic T cell response of NK-based immunotherapy by augmenting antigen-specific CD8⁺ T cell expansion.

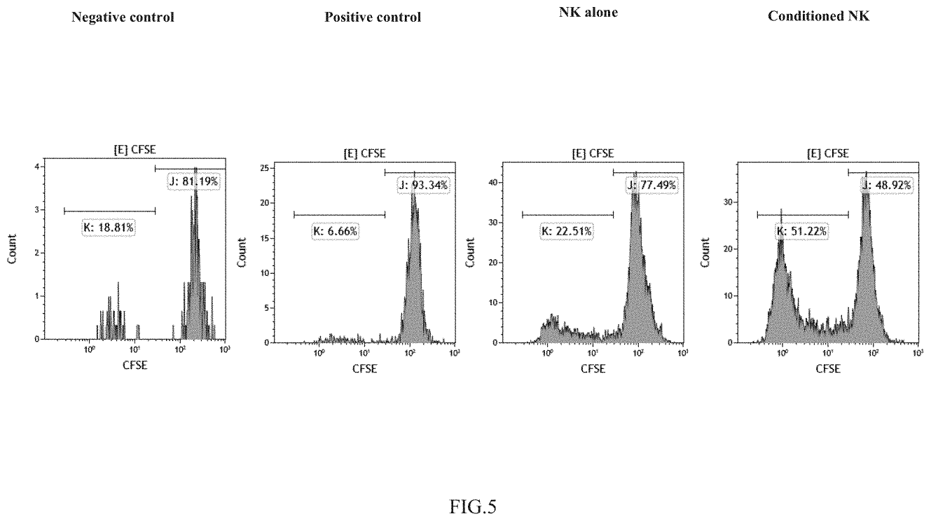

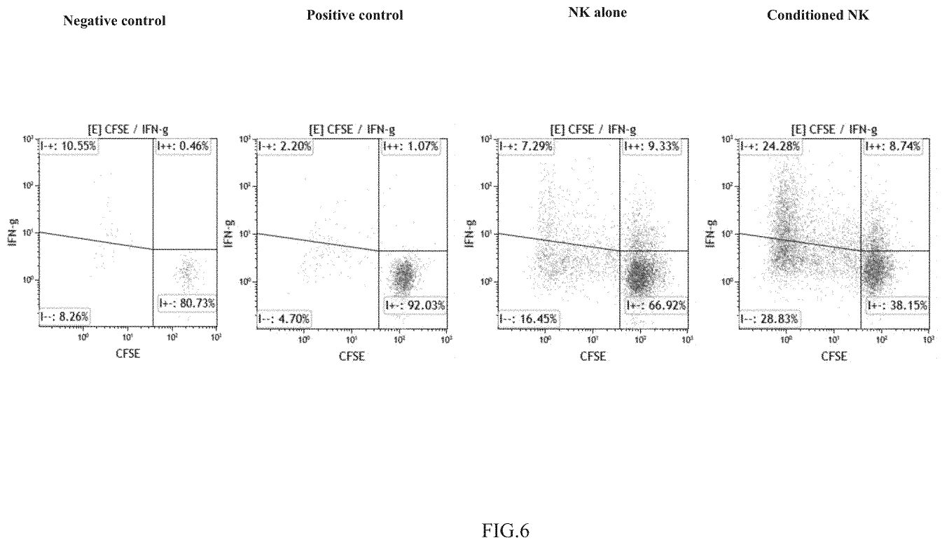

Similarly, as shown in FIG. 5, results of a flow cytometry analysis of splenocytes of the mice are provided. The splenocytes were prepared as described above. CFSE-diluted patterns of lineage⁻TCR_{ab}⁺CD4⁺ T cells and CD8⁺ T cells are determined and shown on dot plots by using specific antibodies and flow cytometry. After re-stimulation, the percentages of antigen-specific CD8⁺ T cells were 22.51% and 51.22% in the NK alone group and conditioned NK group, respectively; in other words, NK-based immunotherapy using β-glucan conditioned NK cells increased tumor antigen-specific CD8⁺ T cells proliferation by nearly 30%, as compared to the immunotherapy without using β-glucan conditioned NK cells. Referring now to FIG. 6, to further elucidate whether the proliferated antigen-specific CD8⁺ T cells were activated, the expression level of interferon-γ (INF-g) was measured in CFSE-diluted CD8⁺ T cells. As shown in FIG. 6 the INF-g level was higher in the conditioned NK group than in the NK alone group. That is, the activation of antigen-specific CD8⁺ T cells was increased by nearly 17% when NK cells were treated with β-glucan prior to infusion. The results demonstrate the capability of β-glucan in enhancing adaptive immunity of the subject receiving the NK-based immunotherapy.

In conclusion, the method of in-vitro activation of immune cells using β-glucan according to the embodiments of the present disclosure improves tumor inhibitory effect of immune cell therapies. Furthermore, infusion of immune cells treated with β-glucan results in enhanced innate and adaptive immunity of the subject receiving the immune cell therapy. Therefore, β-glucan is effective in boosting efficiency and efficacy of immune cell therapies by acting as a potent adjuvant for promoting in vitro expansion and activation of immune cells.

Previous descriptions are only embodiments of the present disclosure and are not intended to limit the scope of the present disclosure. Many variations and modifications according to the claims and specification of the disclosure are still within the scope of the claimed disclosure. In addition, each of the embodiments and claims does not have to achieve all the advantages or characteristics disclosed. Moreover, the abstract and the title only serve to facilitate searching patent documents and are not intended in any way to limit the scope of the claimed disclosure.

What is claimed is:

1. A method for in vitro activation of immune cells, comprising:
contacting a population of immune cells with β-glucan to obtain a population of conditioned immune cells,
wherein when introduced into a subject, the population of conditioned immune cells inhibits tumor growth in the subject.

2. The method according to claim 1, wherein adaptive immunity of the subject induced by the population of conditioned immune cells is stronger than that induced by the population of immune cells.

3. The method according to claim 1, wherein the population of immune cells comprise natural killer (NK) cells.

4. The method according to claim 1, wherein the β-glucan comprises glucose monomers organized as β-(1,3)-linked glucopyranose backbone with periodic β-(1,3) glucopyranose branches linked to the backbone via β-(1,6) glycosidic linkages.

5. The method according to claim 4, wherein the β-glucan is extracted from Saccharomyces cerevisiae.

6. The method according to claim 1, wherein a concentration of the β-glucan falls within a range of 40 μg/ml to 4 mg/ml.

7. The method according to claim 1, wherein the population of immune cells are contacted with the β-glucan for a duration of 12-15 days.

8. The method according to claim 1, further comprising: contacting the population of immune cells with at least one cytokine.

9. The method according to claim 8, wherein the cytokine comprises interleukin-15 (IL-15), interleukin-2 (IL-2), interleukin-12 (IL-12), interleukin-18 (IL-18), and interleukin-21 (IL-21).

10. A population of NK cells activated according to the method of claim 1.

11. A method for inhibiting tumor growth in a subject, comprising:
contacting a population of immune cells with β-glucan to obtain a population of conditioned immune cells; and
administering a therapeutically effective amount of the population of conditioned immune cells to the subject.

12. The method according to claim 11, wherein adaptive immunity of the subject induced by the population of conditioned immune cells is stronger than that induced by the population of immune cells.

13. The method according to claim 11, wherein the population of immune cells comprise NK cells.

14. The method according to claim 11, wherein the β-glucan comprises glucose monomers organized as β-(1-3)-linked glucopyranose backbone with periodic β-(1,3) glucopyranose branches linked to the backbone via β-(1,6) glycosidic linkages.

15. The method according to claim 11, wherein the β-glucan is extracted from Saccharomyces cerevisiae.

16. The method according to claim 11, wherein a concentration of the β-glucan falls within a range of 40 μg/ml to 4 mg/ml.

17. The method according to claim 11, wherein the population of immune cells are contacted with the β-glucan for a duration of 12-15 days.

18. The method according to claim 11, further comprising: contacting the population of immune cells with at least one cytokine.

19. The method according to claim 18, wherein the cytokine comprises IL-15, IL-2, IL-12, IL-18, and IL-21.
